(19) United States
(12) Patent Application Publication
(10) Pub. No.: US 2013/0296969 A1
(43) Pub. Date: Nov. 7, 2013

(54) Electrode Weighting Unit

(71) Applicant: Brett Swanson, St. Ives (AU)

(72) Inventor: Brett Swanson, St. Ives (AU)

(73) Assignee: Cochlear Limited, Sydney (AU)

(21) Appl. No.: 13/464,823

(22) Filed: May 4, 2012

(51) Int. Cl. A61N 1/36; A61F 11/04

(52) U.S. Cl. 607/57

(57) ABSTRACT

The present application discloses systems and methods for performing a weighting operation in a stimulation module of a stimulation prosthesis. Accordingly, a command module transmits one data word (i.e., the channel and amplitude value) to the stimulation module. The stimulation module then determines the individual electrodes and magnitudes of electrical signals to apply thereto so as to achieve appropriate stimulation on the channel. In accordance with one embodiment, a stimulation module includes a receiver that is configured for receiving stimulation data transmitted by the command module, the stimulation data defining a channel and an amplitude, a weighting unit configured for calculating, based on the channel and amplitude value, a set of magnitudes, and an arrangement of current sources-and-switches configured for applying current to the array of electrodes based on the calculated set of magnitudes.

### BACKGROUND

Various types of hearing prostheses may provide persons with different types of hearing loss with the ability to perceive sound. Hearing loss may be conductive, sensorineural, or some combination of both conductive and sensorineural hearing loss. Conductive hearing loss typically results from a dysfunction in any of the mechanisms that ordinarily conduct sound waves through the outer ear, the eardrum, or the bones of the middle ear. Sensorineural hearing loss typically results from a dysfunction in the inner ear, including the cochlea where sound vibrations are converted into neural signals, or any other part of the ear, auditory nerve, or brain that may process the neural signals.

Persons with certain forms of sensorineural hearing loss may benefit from cochlear implants and/or auditory brainstem implants. For example, cochlear implants may provide a person having sensorineural hearing loss with the ability to perceive sound by stimulating the person's auditory nerve via an array of electrodes implanted in the person's cochlea.

Current hearing prostheses and other stimulation prostheses are usually configured with two different modules: a command module, which incorporates a sound processor, and a stimulation module that applies stimulation to the recipient via an array of electrodes. Typically, the electrodes are arranged into groups called channels, with the particular type of channel arrangement depending on the sound coding strategy and the stimulation mode in use. Generally, applying current to one channel results in applying current to the one or more electrodes that are defined by that channel.

In operation, the command module determines particular channels on which to apply current as well as the amplitudes of such current. The command module also determines the individual electrodes and magnitudes of current to apply to those electrodes to achieve the appropriate stimulation on the channel. This is referred to as a “weighting operation.” When phased array stimulation modes are used, the stimulation module applies stimulation to the recipient via every electrode in the electrode array to achieve appropriate stimulation on the channel. This results in the command module transmitting a data word for every electrode, which, in some cases, considerably reduces the pulse rate. The pulse rate refers to the number of individual applications of current to a channel per a unit of time.

### SUMMARY

To help ameliorate this shortcoming, the present application discloses a stimulation prosthesis in which the weighting operation is performed in the stimulation module. Thus, the command module transmits one data word (i.e., the channel and amplitude value) to the stimulation module. The stimulation module, rather than the command module, then determines the individual electrodes and magnitudes of current to apply thereto so as to achieve appropriate stimulation on the channel. Accordingly, in some embodiments, this reduces the amount of data transmitted between the command module and the stimulation modules per pulse, thereby allowing higher pulse rates.

In accordance with one embodiment, a stimulation module includes a receiver that is configured for receiving stimulation data transmitted by the command module, the stimulation data defining a channel and an amplitude, a weighting unit configured for calculating, based on the channel and amplitude, a set of magnitudes, and an arrangement of current sources and switches configured for applying current to the array of electrodes based on the calculated set of magnitudes.

### BRIEF DESCRIPTION OF THE DRAWINGS

FIG. 1 shows a block diagram of an example stimulation prosthesis.

FIG. 2A shows an example weights matrix for 20 tripolar channels and 22 electrodes.

FIG. 2B shows an example weights matrix for 22 phased array channels and 22 electrodes.

FIG. 3 shows a block diagram of an example stimulation module, in accordance with one embodiment.

FIG. 4 shows a block diagram of another example stimulation module, in accordance with one embodiment.

FIG. 5 shows a block diagram of an example weighting unit, in accordance with one embodiment.

FIG. 6A shows a block diagram of an example multiplier-accumulator, in accordance with one embodiment.

FIG. 6B shows a block diagram of an example MAC vector unit, in accordance with one embodiment.

FIG. 7 depicts example program code, in accordance with one embodiment.

FIG. 8 depicts example program code, in accordance with one embodiment.

FIG. 9 depicts example program code, in accordance with one embodiment.

### DETAILED DESCRIPTION

The following detailed description describes various features and functions of the disclosed systems and methods with reference to the accompanying figures. In the figures, similar symbols typically identify similar components, unless context dictates otherwise. The illustrative system and method embodiments described herein are not meant to be limiting. Certain aspects of the disclosed systems and methods can be arranged and combined in a wide variety of different configurations, all of which are contemplated herein.

Certain aspects of the disclosed systems, methods, and articles of manufacture may be described herein with reference to cochlear implant embodiments. However, the disclosed systems, methods, and articles of manufacture are not so limited. Many of the disclosed features and functions described with respect to the cochlear implant embodiments may be equally applicable to other embodiments that may include other types of hearing prostheses, such as, for example, auditory brain stem implants, and prostheses that use both electrical and acoustic stimulation, sometimes referred to as hybrid devices. Further, many of the disclosed features and functions can be applied to any general stimulation prosthesis that includes a data link over which the prosthesis transmits data.

Stimulation Prosthesis Overview

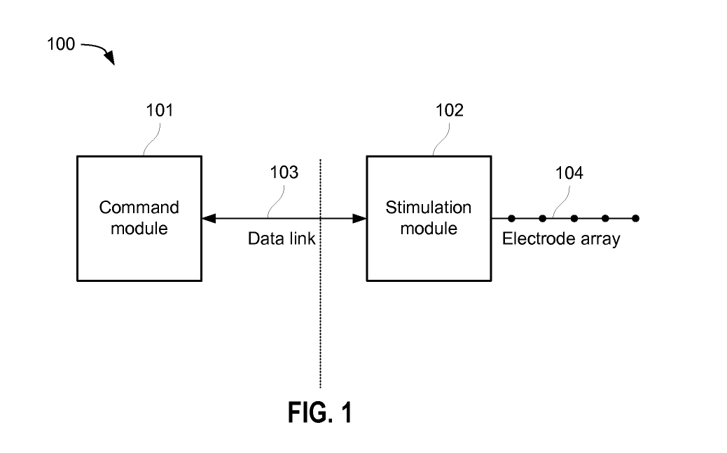

FIG. 1 depicts a stimulation prosthesis 100, which includes a command module 101, and a stimulation module 102, which generates stimulation pulses for an electrode array 104. Typically, the command module 101 includes several components, such as a processor, memory storage and/or a radio frequency (RF) interface. In a cochlear implant system, the command module 101 typically has at least one microphone, and the command module 101 processes an audio signal to determine an appropriate pattern of electrical stimulation, in accordance with a set of rules referred to as a sound coding strategy.

In operation, the command module 101 transmits information specifying the desired stimulation pattern over the data link 103. Typically, data link 103 is a serial, transcutaneous RF inductive data link. In such cases, the flow of data from the command module 101 across data link 103 to stimulation module 102 provides power to stimulation module 102. Generally, the more data that is communicated from command module 101 to stimulation module 102, the more power used by command module 101 and received by stimulation module 102. However, data link 103 may be any wired or wireless data link. In response to receiving the information specifying the desired stimulation pattern, the implanted stimulation module 102 generates electrical stimuli and delivers those stimuli to an array of electrodes 104, which are implanted in a recipient's cochlea or other part of the body, as the case may be. Stimulation prostheses, such as the prosthesis 100 depicted in FIG. 1 may be configured to apply stimulation to any part of the body, such as the brain, the vestibular system (e.g., to treat balance disorders), the muscular efferent system (e.g., to treat paraplegia), or other afferent nerves (e.g., to restore sensory capabilities). As mentioned above, electrically stimulating nerves in a cochlea with a cochlear implant, for example, may enable persons with sensorineural hearing loss to perceive sound.

Example Stimulation Modes

An electrical stimulation generated by the stimulation prosthesis 100 generally produces a flow of electrical current between at least one active electrode and at least one reference electrode. The stimulation mode describes the configuration of the electrodes.

The most commonly used stimulation mode in cochlear implant systems is called “monopolar mode”, in which current flows between an intra-cochlear electrode (the active electrode) and one or more extra-cochlear electrodes (the reference electrodes). In monopolar mode, channels are defined by one active electrode. For instance, a “channel one” is defined by the first electrode in the electrode array 104 and a reference electrode, a “channel two” is defined by the second electrode in the electrode array 104 and a reference electrode, and so on. Thus, in this case, applying stimulation to channel one means applying a current between the first electrode in electrode array 104 and its corresponding reference electrode. Monopolar mode can be advantageous because only a single current source is required to apply one instance of stimulation.

However, one potential drawback associated with monopolar mode is that there is usually a large spread of excitation. Typically, when applying an electrical signal to one monopolar channel, nerve fibers of a recipient's cochlea that are located some distance away from the active electrode may also be excited. Reducing spread of excitation is advantageous because it reduces interference with other channels.

Other example stimulation modes use multiple current sources to deliver stimulation to the recipient's cochlear simultaneously via multiple active electrodes. One such mode is called “bipolar mode.” For instance, applying stimulation via one channel in bipolar mode results in current flowing into (such current is referred to as “negative current”) one active electrode and simultaneously out of another active electrode (such current is referred to as “positive current”). Another such mode is called “tripolar mode.” For example, stimulation applied via one channel in tripolar mode results in current flowing out of a central active electrode while simultaneously flowing into two other active electrodes.

Still another example stimulation mode is called “phased array mode.” Generally, a stimulation module operating in phased array mode will use most, if not all of the electrodes in the electrode array 104 to deliver stimulation to the recipient's cochlea. In phased array mode, each channel calls for electric current to be applied to all of the active electrodes in varying proportions. One advantage of using a stimulation mode that incorporates multiple current sources is that such modes may have a reduced spread of excitation.

Example Weighting Operation

As a general matter, in cases in which the hearing prosthesis applies current to multiple electrodes simultaneously to deliver stimulation to the recipient's cochlea, it becomes helpful to distinguish between the current applied on a channel and individual currents applied to the individual electrodes that correspond to the channel. For brevity's sake, the current applied on a channel will be referred to as having an “amplitude,” whereas the current applied to an individual electrode will be referred to as having a “magnitude.” (These terms are not meant to be limiting in any way and are used solely to avoid ambiguity.)

As described above, one channel may have multiple corresponding electrodes. Thus, those electrodes are said to participate in that channel and that channel is said to contribute to those electrodes. Moreover, one electrode can participate in multiple different channels. Threshold and comfort levels for a particular implant recipient are associated with channels. A channel's threshold level is defined as the smallest amount of current applied to the channel that will result in a noticeable sound sensation by the recipient. A channel's comfort level is defined as the largest amount of current applied on the channel that results in a volume of sound sensation that is still comfortable to the recipient. These values are specific to the recipient and are usually determined at a fitting session with an audiologist or other similarly trained specialist.

In operation, the command module 101 determines a series of pulses, where each pulse is specified by the channel it is to be delivered on, and the amplitude of current to apply. Given a specified amplitude and a specified channel, the prosthesis typically performs a calculation to determine the particular electrodes and the particular magnitudes of current that should be applied on those electrodes. In the general case of applying stimulation with current amplitude i_{c} on a channel c, the magnitude of current iₑ on a participating electrode e can be expressed as:

iₑ=i_{c}W_{ce}

where W is a weights matrix, and each element W_{ce} is the contribution of channel c to electrode e.

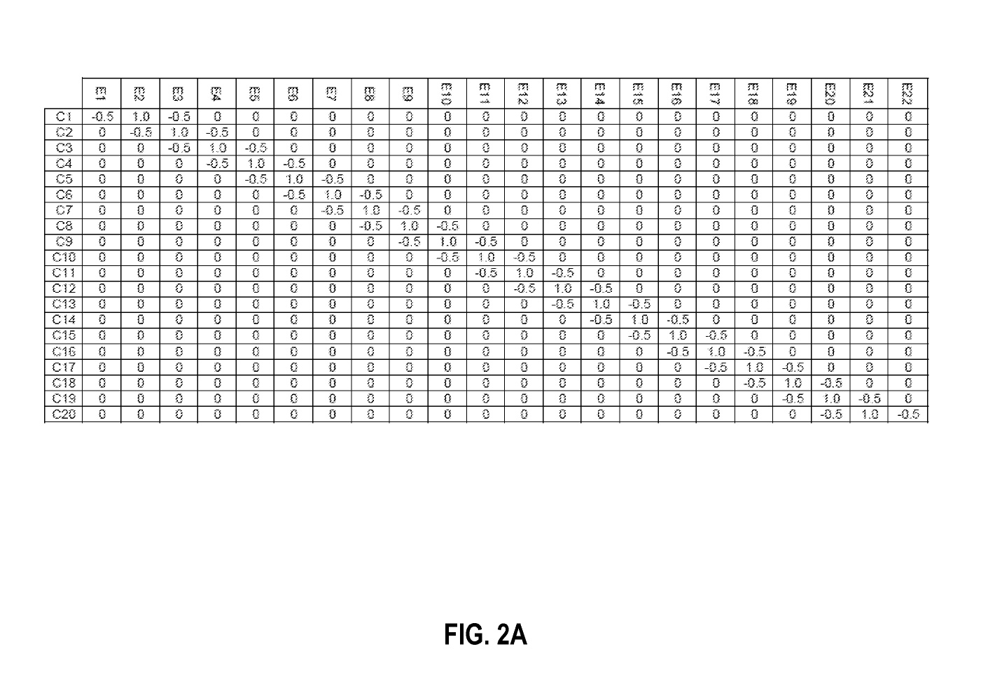

FIG. 2A depicts an example weights matrix W for operation according to the tripolar mode with an electrode array having 22 electrodes. The individual electrodes of the array are labeled across the top, E1 to E22. Each row of the matrix defines one tripolar channel and includes weights for every electrode in the electrode array; in tripolar mode, each row contains three non-zero weights. Each row in the matrix is also sometimes referred to as a vector of electrode weights. In this example, there are 20 tripolar channels, labeled C1 to C20.

FIG. 2B depicts an example weights matrix W for operation according to phased array mode with an electrode array having 22 electrodes. The individual electrodes of the array are labeled across the top, E1 to E22. Each row of the matrix defines one phased array channel and includes weights for every electrode in the electrode array; in phase array mode, every weight may be non-zero. In this example, there are 22 phased array channels, labeled C1 to C22. The matrices depicted in FIGS. 2A and 2B are example weights matrices, and in other cases, other weights matrices can be used, including weights matrices with different weights, different configurations of channels, more or fewer channels, and/or more or fewer electrodes.

In one example that uses the weights matrix depicted in FIG. 2A, channel C4 includes three electrodes: E4, E5, and E6. Those electrodes have non-zero weights, whereas the remaining electrodes have zero weights. To apply a current with an amplitude of 100 μA to channel C4, the stimulation module 102 will apply (i) a current magnitude of −0.5*100 μA=−50 μA on electrode E4, (ii) a current magnitude of +1.0*100 μA=+100 μA on electrode E5, and (iii) a current magnitude of −0.5*100 μA=−50 μA on electrode E6. The stimulation pattern produced by this channel C4 is centered on E5, and typically has a smaller spread of excitation than that which results from delivering current on electrode E5 in a monopolar mode.

The general process of calculating the specific electrodes and the specific magnitudes of current that should be applied to those electrodes based on a specified channel and a specified amplitude is called a “weighting operation.” When the command module 101 carries out the weighting operation, then the command module 101 transmits to the stimulation module a data word indicating the current magnitude to apply to each electrode. Thus, in accordance with the example provided above, the command module 101 performs the weighting operation and transmits to the stimulation module (i) a data word indicating to apply −50 μA on electrode E4, (ii) a data word indicating to apply +100 μA on electrode E5, and (iii) a data word indicating to apply −50 μA on electrode E6. Moreover, in a prosthesis that utilizes phased array mode, all entries of the weights matrix may be non-zero. Thus, current applied on a single channel results in the stimulation module applying current to all electrodes in the electrode array.

One example calculation used to produce a specified amplitude on a specified channel is expressed in the Python language as:
for e in range(num_electrodes):

e1_mag_vec[e]=amplitude*weight_matrix[channel, e]

where e1_mag_vec is the output vector of current magnitudes, and weight_matrix is the weights matrix. Here, the amplitude is multiplied by one row of the weights matrix. In prostheses that use phased array mode, performing the weighting calculation in the command module 101 results in twenty-two magnitude words per pulse sent over the data link.

In some prostheses, it is also useful to deliver stimulation to the recipient by applying current to multiple channels simultaneously. In such prostheses, the magnitude of current applied on a given electrode is calculated as the sum of the magnitudes that each channel contributes to the given electrode. One example calculation to produce current on multiple channels simultaneously can be expressed in the Python language as:

e1_mag_vec=zeros((num_electrodes))
for c in range(num_channels):
   for e in range(num_electrodes):

e1_mag_vec[e]+=ch_amp_vec[c]*weight_matrix[c, e]

where ch_amp_vec is the input vector of channel amplitudes, e1_mag_vec is the output vector of electrode magnitudes, and weight_matrix is the weights matrix. This can be written as a matrix multiply command, in the form of:

e1_mag_vec=ch_amp_vec*weight_matrix.

Example Stimulation Module Configuration and Operation

In one embodiment of the present invention, the stimulation module 102 is configured to perform the weighting operation. In this embodiment, the command module 101 specifies, for each pulse, a channel and an amplitude (for example, in accordance with a particular sound coding strategy), and transmits the channel and amplitude across the data link 103 to the stimulation module 102. The stimulation module then performs the weighting operation to determine which individual electrodes to use and the magnitudes of current to apply to those electrodes.

In this embodiment, carrying out the weighting operating at the stimulation module 102 instead of at the command module 101 reduces the amount of data transmitted across the data link 103. For instance, in prostheses that use the phased array stimulation mode on twenty-two electrodes and carry out the weighting operation at the command module 101, twenty-two magnitude words are sent across the data link per pulse. However, in embodiments of the invention that use phased array on twenty-two electrodes, the weighting operation is carried out at the stimulation module 102, and the command module sends only one amplitude word across the data link per pulse.

Reducing the amount of data transmitted across data link 103 is advantageous, especially in embodiments that use a transcutaneous RF inductive data link because it reduces the amount of power consumption. Where the power is derived from a battery or other finite power source, reducing power consumption often translates into longer battery life, which is generally advantageous.

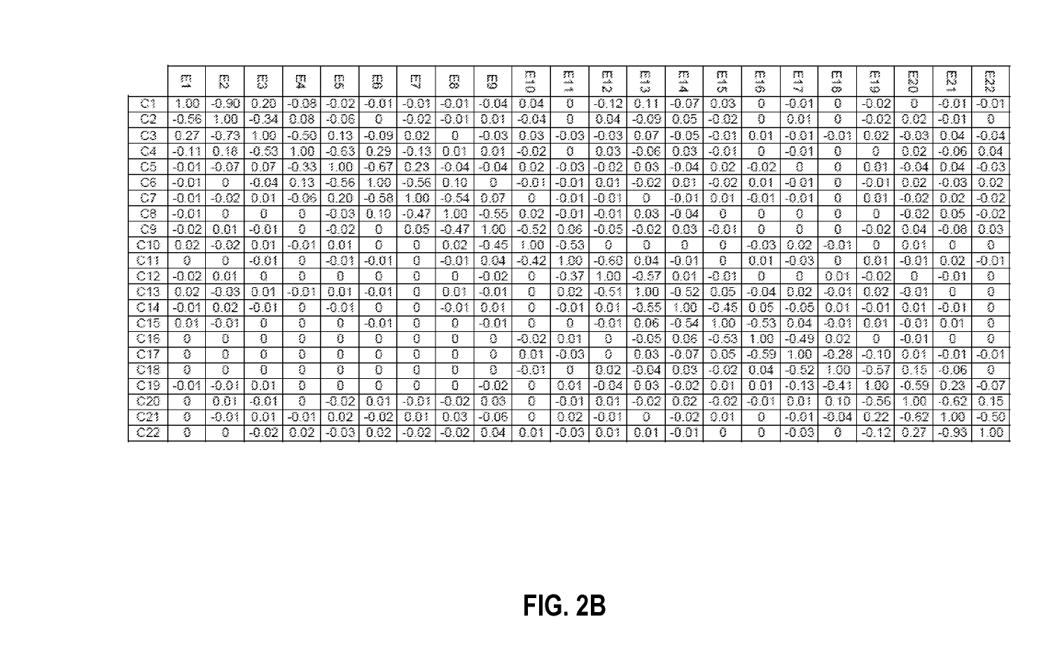

FIG. 3 depicts a block diagram of a stimulation module 311 according to one example embodiment. Stimulation module 311 includes an RF Receiver 301 configured to receive an RF signal 300 from the command module 101 across data link 103 (FIG. 1). The RF receiver 301 is further configured to output a serial data stream 302 to a Protocol Decoder 303. Protocol Decoder 303 is configured to decode the received data stream 302 according to a particular protocol and data format in use. Protocol decoder 303 is further configured to provide channel data 304 to a Weighting Unit 305. In some embodiments, Channel data 304 specifies the stimulation pulses in terms of channel indices and channel amplitudes.

In one embodiment, the Weighting Unit 305 stores a weights matrix and performs calculations to determine magnitudes of current to apply to certain electrodes. Weighting unit 305 outputs the result of these calculations as electrode data 306. The electrode data 306 is used by the Pulse State Machine 307 to generate the control signals 308 for use by Current Sources and Switches 309 to produce stimulation currents 310 having the desired magnitude on the desired electrodes.

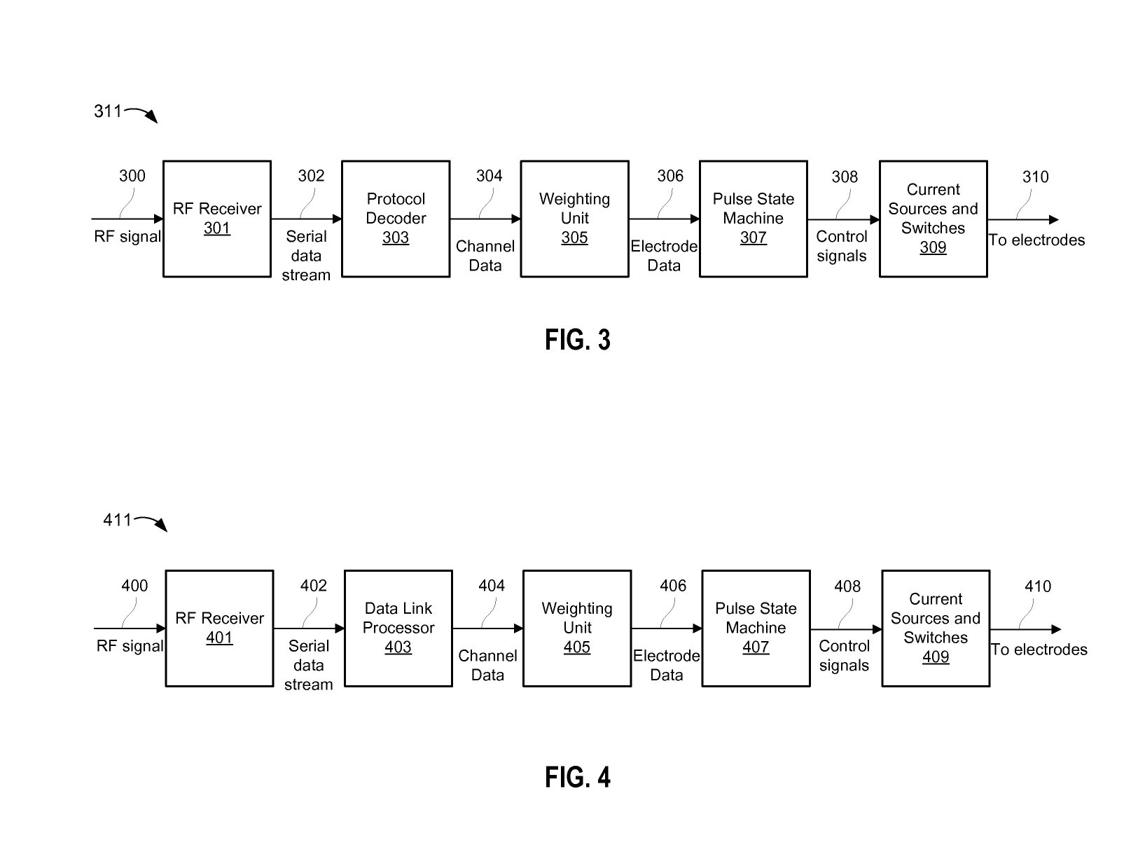

One purpose of the Protocol Decoder 303 is to extract from the data stream 302 the channel and the amplitude for each pulse. The Protocol Decoder 303 delivers this information to the Weighting Unit 305 at the appropriate time. FIG. 4 depicts another stimulation module 411 according to one example embodiment. Stimulation module 411 is similar to stimulation module 311 and therefore is not described in particular detail. One difference, however, is that Protocol Decoder 303 is depicted as a Data Link Processor 403. Generally, Data Link Processor 403 is a programmable processor and its functionality is therefore determined by software.

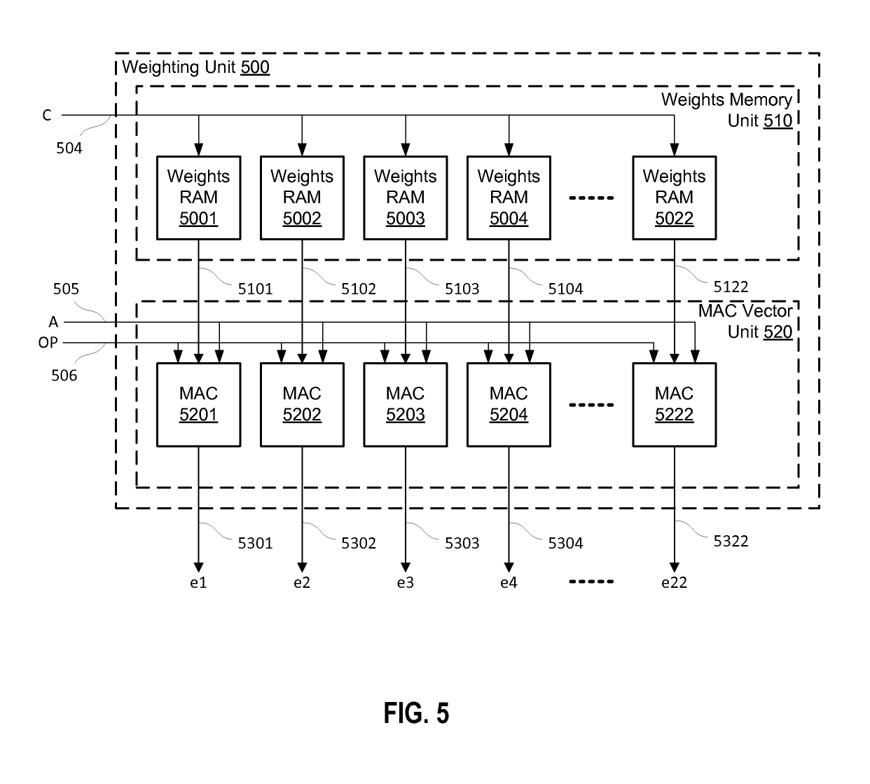

FIG. 5 depicts an example Weighting Unit 500 that in some embodiments is configured to carry out a weighting operation. Embodiments that utilize weighting unit 500 to carry out a weighting operation use less power than embodiments that utilize a programmable microprocessor or other entity with functionality determined by software. Again, using less power is advantageous for one reason because it typically will yield longer battery lives. Moreover, in some embodiments, weighting unit 500 uses less space within a stimulation module than does a programmable microprocessor. Using less space is advantageous because it allows the stimulation module to be smaller and less intrusive. Other advantages may become evident as well given the present disclosure.

Weighting Unit 500 comprises Weights Memory Unit 510 and MAC Vector Unit 520. Both Weights Memory Unit 510 and MAC Vector Unit 520 are divided into sections, with one section for each electrode in the electrode array. FIG. 5 depicts an embodiment of Weighting Unit 500 with twenty-two sections for an electrode array with twenty-two electrodes. Other embodiments have more or fewer sections, depending on how many electrodes are used in those embodiments' electrode arrays.

Weights Memory Unit 510 stores the weights matrix W discussed above. Weights Memory Unit 510 comprises Weights RAMs 5001-5022. Each Weights RAM stores the weights for one electrode. In an embodiment that utilizes one of the weights matrices W discussed above with respect to FIGS. 2A and 2B, each Weights RAM stores one column of the weights matrix W. In particular, Weights RAM 5001 stores the first column of weights matrix W, Weights RAM 5002 stores the second column of weights matrix W, and so on. The number of entries in each column is at least equal to the number of channels. In one embodiment, a stimulation module, such as stimulation module 411, 311, or 102, receives a weights matrix from command module 101 (or another entity) and partitions the matrix into separate columns for each electrode. The stimulation module then stores the columns in the individual Weights RAMs in the Weights Memory Unit 510. The Weights RAMs may be provisioned with weights in other ways as well.

Weighting Unit 500 also includes an address bus 504, labeled C, which is used as an address bus for each Weights RAM 5001-5022. Whenever a pulse is desired, Protocol Decoder 303 (FIG. 3) or Data Link Processor 403 (FIG. 4) provides the channel number of the pulse on address bus 504. The channel number is used to select one value of the column stored in each weights RAM. Each Weights RAM then outputs the selected value onto its corresponding output bus (5101-5122). Thus the set of values that appear on weights RAM output busses 5101-5122 represent one row of the weights matrix, corresponding to the channel number, C. This set of values represent the weight that channel C contributes to each electrode. In Python syntax, for example, this set of values can be denoted weight_matrix[c,:].

For instance, in an embodiment in which the Weights Memory Unit 510 stores the weights matrix depicted in FIG. 2A, weights RAM 5022 stores the column of the weights matrix associated with electrode 22 (i.e. the right-most column as depicted). When a pulse on channel 20 is desired, address bus 504 carries the value c=20, and weights RAM 5022 outputs the value −0.5 onto output bus 5122, which is the contribution of channel 20 to electrode 22.

Because, in the embodiment depicted in FIG. 5, all weights RAMs share the same address bus 504, another embodiment is possible in which Weights Memory Unit 510 includes a single memory with a wide memory word, where each memory word holds one row of the weights matrix. Other configurations of Weights memory Unit 510 are possible as well.

In one embodiment, as depicted in FIG. 5, MAC Vector Unit 520 comprises a set of MACs 5201-5222. The output bus of a given MAC carries a magnitude word for the corresponding electrode, denoted en, where n is the electrode number. For example, MAC 5201 generates magnitude word 5301 for electrode 1, labeled e1; MAC 5202 generates magnitude word 5302 for electrode 2, labeled e2; and so on. In one embodiment, the number of bits in each magnitude word is equal to the number of bits of the digital-to-analog converter (DAC) used in the current source, examples of which range from 8 to 12 bits; however, any values are possible. The set of magnitude words (i.e., e1 to e22 in this example) is similar to the electrode magnitude vector, described above, an example of which is given by the following expression

e1_mag_vec=[e1, e2, e3, e4, . . . , e22]

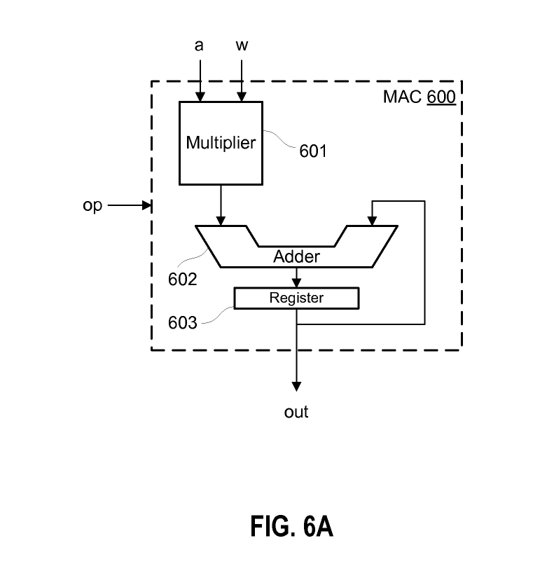

MAC Vector Unit 520 also includes a signal bus 505, labeled A, that carries an indication of a channel amplitude, and a signal bus 506, labeled OP, that carries an opcode, i.e. an indication of a desired operation to perform. The signals on these busses are delivered to each MAC (5201-5222). FIG. 6A depicts a block diagram of an example MAC 600. MAC 600 includes a multiplier 601, an adder 602, and a magnitude register 603 for storing an electrode magnitude output. In one embodiment, multiplier 601 is a parallel multiplier; such a multiplier may be configured to perform the multiply operation in a single clock cycle. In other embodiments, multiplier 601 consumes less area and power, but takes more clock cycles to perform the multiply operation.

Returning to FIG. 5, the signal bus OP specifies an operation to be performed by MAC Vector Unit 520. In one embodiment, there are three possible operations, each of which is described in Table 1. In other embodiments however, different operations are possible as well.

TABLE 1

| | | Assembly | |
|---|---|---|---|
| Opcode | MAC operation | syntax | Description |
| CLR_E | out = 0 | E = 0 | Clear electrode magnitudes. |
| MUL_E | out = a * w | E = | Multiply amplitude by weights |
| | | a * W[c,:] | for channel c. |
| MAC_E | out += a * w | E += | Multiply amplitude by weights |
| | | a * W[c,:] | for channel c and accumulate. |

In one embodiment, these operations are implemented as instructions of the Data Link Processor 403, and are referred to as “vector” instructions because they operate on each of the MACs 5201-5222 in MAC Vector Unit 520 in parallel. The assembly syntax uses the symbol E to represent the vector [e1, e2, e3, e4, . . . , e22]. As described in Table 1, when the opcode CLR_E is executed, all magnitude registers in MACs 5201-5222 are cleared. When the opcode MUL_E is executed, the channel number appears on the C bus 504, and is used to address each of the Weights RAMs 5001-5022. The outputs 5101-5122 of each of the weights RAM, taken together, correspond to one row of the weights matrix W (i.e., the weights of each electrode participating in the specified channel). This is represented in assembly syntax as W[c,:]. This row vector is multiplied by the value on the A bus 505 in each multiplier 601 of the MACs, and each product is passed through each adder 602 and stored in each magnitude register 603, thus appearing as magnitude vector E on output lines 5301-5322. When the opcode MAC_E is executed, the channel number appears on the C bus 504 (similarly to the MUL_E opcode) and is used to address each of the Weights RAMs, and the corresponding row of the weights matrix W is multiplied by the value on the A bus 505 in each multiplier 601 of the MACs. Each adder 602 adds the product to the existing value in each corresponding magnitude register 603.

FIG. 6B depicts another embodiment of a MAC Vector Unit. In the embodiment depicted, MAC Vector Unit 700 uses a shift-accumulate method to perform multiplications. The inputs to MAC Vector Unit 700 in FIG. 6B are similar to the inputs to MAC Vector Unit 520 in FIG. 5, and include A bus 505, OP bus 506 and Weights RAM outputs 5101-5103. MAC Vector Unit 700 includes amplitude shift register 802 (labeled ASH), a set of shift-accumulator units 701-703, and associated control circuitry (not shown in FIG. 6B). There is one shift-accumulator unit for each electrode. For brevity, FIG. 6B depicts only three shift-accumulator units, but a typical embodiment has twenty-two shift-accumulator units for an electrode array with twenty-two electrodes. Signal 803 carries the least significant bit of ASH register 802 to each shift-accumulator unit 701-703. Each shift-accumulator unit (e.g. 701) includes a weight shift register (e.g. 721, labeled WSH1), an adder (e.g. 741), and an electrode magnitude register (e.g. 761, labeled E1). The number of bits in the amplitude A, carried on bus 505, is here denoted NA. The number of bits in the weights, carried on busses 5101-5103, is denoted NW. Register 802 (ASH) has NA bits. Registers 721-723 (WSH1-WSH3) and registers 761-763 (E1-E3) have NA+NW bits. The output of the MAC Vector Unit 700 is the set of electrode magnitude register outputs (771-773), which represent a vector of electrode magnitudes.

MAC Vector Unit 700 is able to perform the operations listed in Table 1, as well as perhaps other operations. Such operations are described in terms of three micro-operations: M_CLR, M_LOAD, and M_SHAC. In some embodiments, each micro-operation takes one clock cycle. When the micro-operation M_CLR is executed, the electrode magnitude registers 761-763 (E1-E3) are cleared. When the micro-operation M_LOAD is executed, the amplitude A, carried on bus 505, is loaded into register 802 (ASH); and the weights, carried on busses 5101-5103, are loaded into registers 721-723 (WSH1-WSH3).

When the micro-operation M_SHAC is executed, the shift-accumulator units 701-703 examine signal 803 (the least significant bit of register 802 ASH), and conditionally accumulate WSH1-WSH3 into E1-E3. For example, in shift-accumulate unit 701, if signal 803 is “one”, then adder 741 adds the contents of register 721 (WSH1) to the contents of register 761 (E1), storing the result back into register 761 (E1); conversely, if signal 803 is “zero,” then no addition is performed, and register 761 (E1) retains its previous value. In the same clock cycle, amplitude shift register 802 ASH is shifted right by one bit, discarding the previous least significant bit. In the same clock cycle, each weight shift register 721-723 (WSH1-WSH3) is shifted left by one bit. Other example executions are possible as well.

The execution of the opcodes described in Table 1 will now be explained further with respect to operation in accordance with one embodiment. For example, Opcode CLR_E invokes the execution of one micro-operation, M_CLR, and thus takes one clock cycle. Opcode MAC_E invokes NA+1 clock cycles: in the first clock cycle, micro-operation M_LOAD is executed; then in the subsequent NA clock cycles, micro-operation M_SHAC is executed. Opcode MUL_E also invokes NA+1 clock cycles: in the first clock cycle, micro-operations M_LOAD and M_CLR are executed simultaneously; then in the subsequent NA clock cycles, micro-operation M_SHAC is executed.

Referring back to the stimulation module 411 of FIG. 4, operation of this stimulation module according to one embodiment involves using a sound coding strategy that delivers stimulation to the recipient's cochlea by applying currents to channels sequentially (i.e., one at a time). In such a sound coding strategy, a current applied to any one channel may involve more than one electrode. One example of the data format used by command module 101 and stimulation module 102 to communicate over the data link 103 is called the “Channel-amplitude format.”

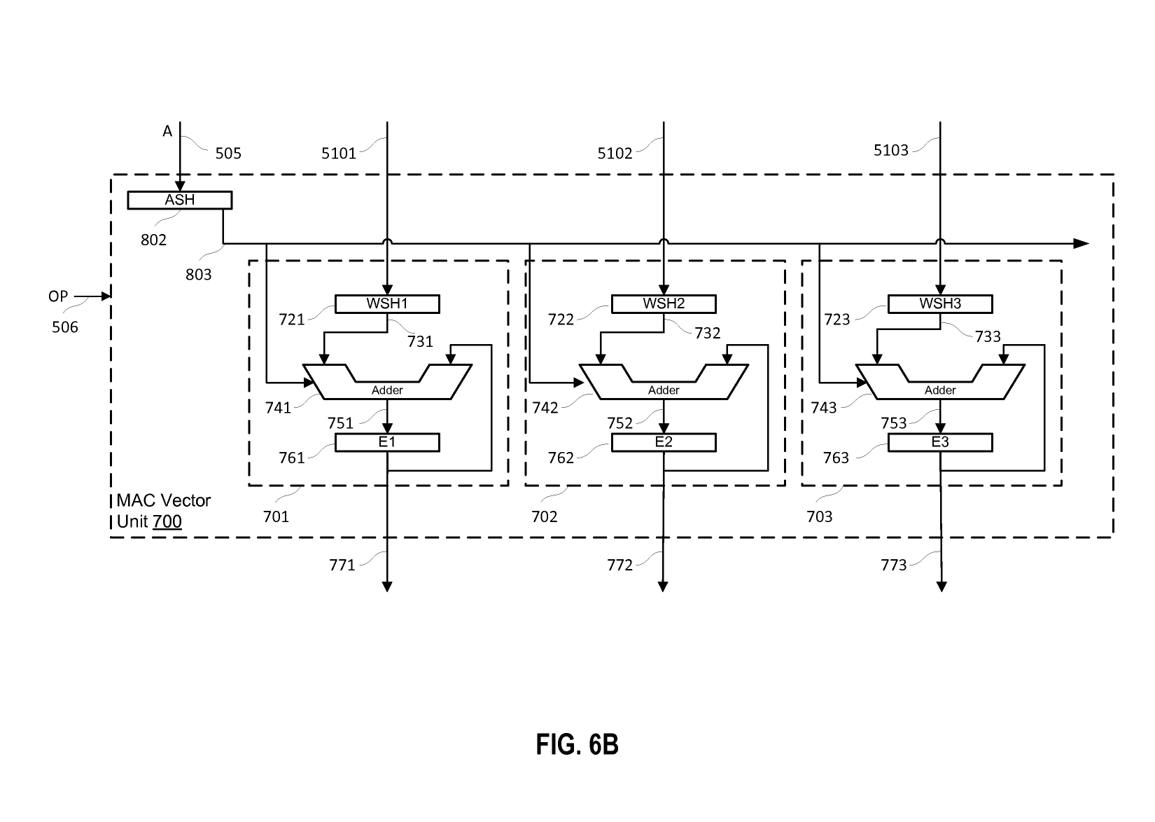

As mentioned above, in one embodiment, during configuration, weights are stored into the Weights RAMs. And during stimulation, the Data Link Processor 403 executes a main loop program, such as the example program code 700 depicted in FIG. 7. Here, the 5-bit channel number is read into register C, and the 8-bit channel amplitude is read into register A. The MUL_E instruction (denoted by the assembly syntax: E=a*W[c,:]) then causes the Weighting Unit to calculate the vector of 22 electrode magnitudes. Further, the pulse instruction causes these 22 electrode magnitudes to be transferred to the Pulse State Machine 407, which drives the Current Sources and Switches to produce a current on each electrode having a magnitude that is determined by the magnitude calculated in the Weighting Unit. In one embodiment, the shape of the current applied to an electrode is a biphasic current pulse; however, other shapes are possible. With the appropriate weights matrix stored in the Weights RAMs, this program code 700 can be used for any stimulation mode (e.g., monopolar, bipolar, tripolar, or phased array). In addition, other examples of main loop code are possible as well.

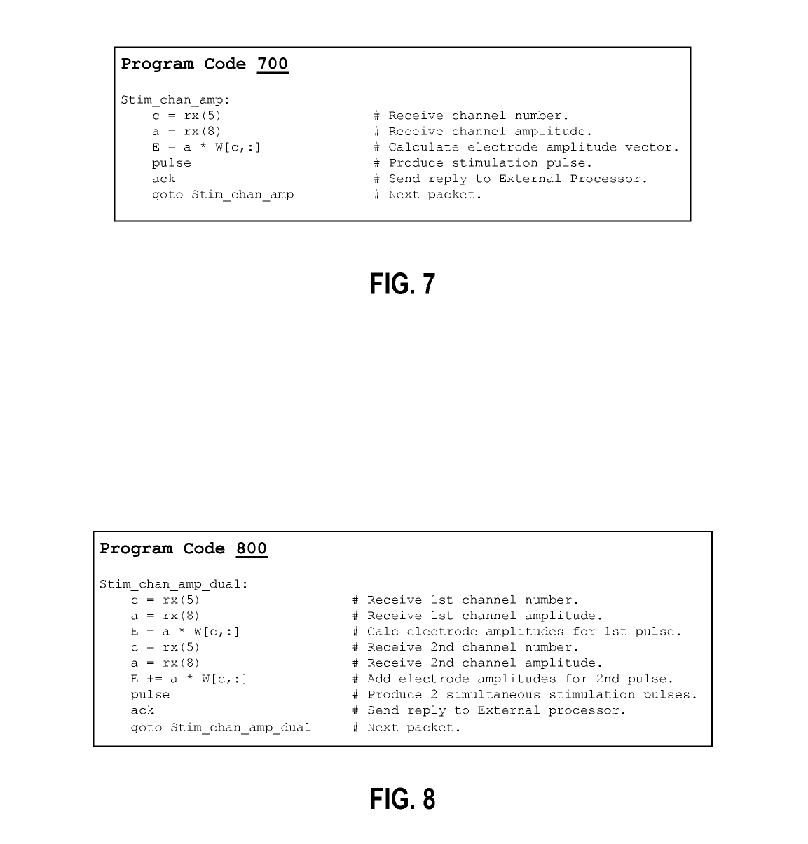

FIG. 8 depicts example program code for applying pulses to two channels at the same time, sometimes referred to as “paired pulsatile stimulation” (PPS). In essence, the stimulation module receives a first channel number and a first amplitude. The MUL_E instruction (assembly syntax: E=a*W[c,:]) causes the Weighting Unit to multiply the first amplitude with the vector of weights corresponding to the first channel. This results in the MAC registers holding a first vector of magnitudes, where each element of the vector holds the contribution of the first channel to the corresponding electrode. Sometime later, the stimulation module receives a second channel number and a second amplitude. The MAC_E instruction (assembly syntax: E+=a*W[c,:]) causes the Weighting Unit to multiply the second amplitude with the vector of weights corresponding to the second channel, and to add the resulting vector to the MAC registers. This results in the MAC registers holding a vector of magnitudes, where each element of the vector holds the sum of the contributions of the first and second channels to the corresponding electrode. After this, the stimulation module transmits this summed vector to the current sources and switches for application to the electrode array. In other embodiments, other examples of program code are possible for applying pulse on multiple channels simultaneously.

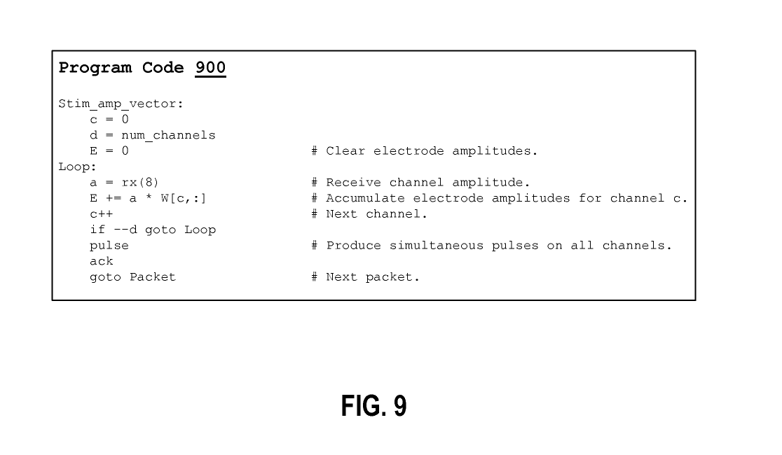

Finally, FIG. 9 depicts the assembly code for a strategy that outputs currents simultaneously on all channels. The stimulation module begins by clearing the MAC registers. Then for each channel, the stimulation module receives an amplitude value, multiplies it by the corresponding row in the weights matrix, and accumulates the resulting vector in the MAC registers. The stimulation module then transmits the final summed vector to the current sources and switches for application to the electrode array. In one embodiment, this example program code is used with phased array weights. Other example program codes are possible as well.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

1-9. (canceled)

10. A method comprising:
a stimulation module receiving stimulation data that specifies (i) a channel and (ii) an amplitude;
based on the specified channel and amplitude, the stimulation module calculating a set of magnitudes, wherein calculating a set of magnitudes includes, based on the specified channel, determining a vector of electrode-weight values, each individual electrode-weight value of the vector of electrode-weight values corresponding to one of the individual electrodes in the array of electrodes, and multiplying the specified amplitude by the vector of electrode-weight values; and
based on the calculated set of magnitudes, the stimulation module applying current to a subset of an array of electrodes, the subset being defined by the specified channel,
wherein the stimulation module includes a multiplier accumulator (MAC) vector unit that includes an amplitude-shift register and a plurality of shift-accumulator units, each shift-accumulator unit including a weight-shift register, an adder, and a magnitude register.

11. The method of claim 10, wherein
each individual electrode-weight value of the vector of electrode-weight values being distinguishable from each other individual electrode-weight value of the vector of electrode-weight values.

12. The method of claim 10,
wherein multiplying the specified amplitude by the vector of electrode-weight values comprises the amplitude-shift register receiving and storing an amplitude value, and each shift-accumulator unit of the plurality of shift-accumulator units receiving and storing into the weight-shift register an individual electrode-weight value, and engaging in an iterative process in which one iteration of the process includes accumulating the value of the weight-shift register into the magnitude register when the least significant bit of the amplitude-shift register is high, doing nothing with the value of the weigh-shift register with respect to the magnitude register when the least significant bit of the amplitude-shift register is low, shifting the amplitude-shift register one bit to the right, and shifting each individual electrode-weight register one bit to the left.

13. The method of claim 12, further comprising:
the stimulation module receiving configuration data that specifies a weights matrix, the weights matrix including a plurality of electrode-weight vectors, wherein determining a vector of electrode-weight values comprises determining which electrode-weight vector from the weights matrix is defined by the specified channel.

14. The method of claim 10, further comprising:
the stimulation module receiving stimulation data that specifies (iii) a second channel, and (iv) a second amplitude, and wherein calculating a set of magnitudes comprises:
based on the specified channel, determining a first vector of electrode-weight values, each individual electrode-weight value of the first vector of electrode-weight values corresponding to one of the individual electrodes in the array of electrodes,
based on the specified second channel, determining a second vector of electrode-weight values, each individual electrode-weight value of the second vector of electrode-weight values corresponding to one of the individual electrodes in the array of electrodes,
multiplying the specified amplitude by the first vector of electrode-weight values, thereby producing a first resultant vector of magnitudes,
multiplying the specified second amplitude value by the second vector of electrode weights to produce a second resultant vector of magnitudes and accumulating the second resultant vector of magnitudes with the first resultant vector of magnitudes.

15. The method of claim 10, wherein the stimulation data is received from a command module that is communicatively coupled to the stimulation module.

16-21. (canceled)

22. A stimulation device comprising:
a command module configured for transmitting data across a data link; and
a stimulation module coupled to an array of electrodes, the stimulation module configured for:
receiving stimulation data transmitted by the command module, the stimulation data defining a channel and an amplitude,
calculating, on a per electrode basis and based on the channel and amplitude value, a set of magnitudes of current to apply to individual electrodes associated with the channel, and
applying current to the array of electrodes based on the calculated set of magnitudes.

23. The stimulator device of claim 22, wherein the stimulation module comprises:
a receiver configured for carrying out the receiving step;
a weighting unit configured for carrying out the calculating step; and
an arrangement of current sources and switches configured for carrying out the applying step.

24. The stimulation device of claim 23, wherein the weighting unit is further configured for:
receiving an indication of the channel from the receiver;
receiving an indication of the amplitude from the receiver;
determining a vector of electrode weights based on the received indication of the channel, each individual electrode weight of the vector of electrode weights corresponding to one of the electrodes of the array of electrodes and being distinguishable from each other individual electrode-weight value of the vector of electrode-weight values; and
multiplying the amplitude by the vector of electrode weights, thereby producing a first resultant vector of magnitudes.

25. The stimulation device of claim 24, wherein the weighting unit is further configured for:
receiving an indication of a second channel;
receiving an indication of a second amplitude;
determining a second vector of electrode weights based on the received indication of the second channel, each individual electrode weight of the second vector of electrode weights corresponding to one of the electrodes of the array of electrodes; and
multiplying the second amplitude value by the second vector of electrode weights to produce a second resultant vector of magnitudes and accumulating the second resultant vector of magnitudes with the first resultant vector of magnitudes, thereby producing a summed vector of magnitudes.

26. The stimulation device of claim 23, wherein the weighting unit further includes a weights memory unit, configured for:
storing a matrix of electrode weights,
receiving an indication of a channel, and
producing a vector of electrode weights, each individual electrode weight of the vector of electrode weights corresponding to one of the electrodes of the array of electrodes and being distinguishable from each other individual electrode-weight value of the vector of electrode-weight values; and
wherein the weighting unit also includes a multiplier-accumulator (MAC) vector unit, configured for multiplying an amplitude by the vector of electrode weights to produce a vector of magnitudes.

27. The stimulation device of claim 26, wherein the MAC vector unit is further configured for calculating a second vector of magnitudes and accumulating the second vector of magnitudes with the first vector of magnitudes, thereby producing a summed vector of magnitudes.

28. The stimulation device of claim 27, wherein the MAC vector unit comprises a plurality of MACs, each individual MAC corresponding to an individual electrode of the array of electrodes, wherein each individual MAC is configured for multiplying an amplitude by an electrode weight to produce a magnitude.

29. The stimulation device of claim 26, wherein the MAC vector unit comprises:
an amplitude-shift register, configured for receiving an amplitude value; and
a plurality of shift-accumulator units, each individual shift-accumulator unit corresponding to one of the electrodes of the array of electrodes, and wherein each individual shift-accumulator includes:
a weight-shift register, configured for receiving an electrode weight value;
an adder, and
a magnitude register; and
wherein the MAC vector unit is further configured to engage in an iterative process in which one iteration of the process includes:
accumulating the value of each individual weight-shift register into the corresponding magnitude register when the least significant bit of the amplitude-shift register is high,
doing nothing with the value of the weigh-shift register with respect to the magnitude register when the least significant bit of the amplitude-shift register is low,
shifting the amplitude-shift register one bit to the right, and
shifting each individual weight-shift register one bit to the left.

30. The stimulation device of claim 23, wherein the command module is further configured for transmitting configuration data that specifies a weights matrix, wherein the receiver is further configured for receiving the configuration data, and wherein the weighting unit is further configured to:
store the weights matrix in the weighting unit, and
based on (i) the channel, (ii) the amplitude value, and (iii) the weights matrix, calculate the set of magnitudes.

31. A stimulation module comprising:
memory storage;
at least one processor; and
program code stored in the memory storage, wherein the program code is executable by the processor to carry out functions comprising:
receiving stimulation data indicating a channel and an amplitude,
based on the indicated channel and amplitude and on a per electrode basis, calculating a set of magnitudes of current to apply to individual electrodes associated with the channel, and
applying a set of currents to an array of electrodes based on the calculated set of magnitudes.

32. The stimulation module of claim 31, wherein applying the set of currents to the array of electrodes comprises applying the set of currents to a subset of two or more electrodes of the array of electrodes.

33. The stimulation module of claim 31, wherein the functions further comprise:
determining a plurality of weight values based on the indicated channel;
multiplying the indicated amplitude by each individual weight value of the plurality of weight values, thereby producing a plurality of magnitudes; and
for each individual magnitude of the plurality of magnitudes, applying to a different individual electrode of the array of electrodes a current based on the individual magnitude.

34. The stimulation module of claim 33,
wherein the functions further comprise receiving configuration data that includes a weights matrix, the weights matrix including a plurality of electrode-weight vectors, and
wherein determining a plurality of weight values based on the indicated channel comprises determining which electrode-weight vector from the weights matrix is defined by the indicated channel.

35. The stimulation module of claim 31, wherein the functions further comprise:
receiving additional stimulation data indicating a second channel and a second amplitude;
determining a plurality of first weight values based on the indicated channel;
multiplying the indicated amplitude value by each individual weight value of the plurality of first weight values, thereby producing a first vector of magnitudes;
determining a plurality of second weight values based on the indicated second channel;
multiplying the second indicated amplitude by each individual weight value of the plurality of second weight values, thereby producing a second vector of magnitudes;
summing the first vector and second vectors, thereby producing a summed vector of magnitudes; and
for each individual magnitude of the summed vector of magnitudes, applying to a different individual electrode of the array of electrodes a current based on the individual magnitude.

36. The stimulation module of claim 31, wherein the processor is a data-link processor configurable by instructions received from a command module.
